(19) United States
(12) Patent Application Publication
(10) Pub. No.: US 2005/0159604 A1
(43) Pub. Date: Jul. 21, 2005

(54) Asymmetric hydrogenation of alpha-amino carbonyl compounds

(75) Inventor: Xumu Zhang, State College, PA (US)

Correspondence Address: Paul D. Greeley, Esq.;Ohlandt, Greeley, Ruggiero & Perle, L.L.P., 10th Floor, One Landmark Square, Stamford, CT, 06901-2682, US

(73) Assignee: The Penn State Research Foundation

(21) Appl. No.: 11/036,157

(22) Filed: Jan. 14, 2005

Related U.S. Application Data
(60) Provisional application No. 60/536,637, filed on Jan. 15, 2004.

(51) Int. Cl. C07D 209/48

(52) U.S. Cl. 548/478; 564/355; 562/444

(57) ABSTRACT

A process for preparing a non-racemic aminoalcohol is provided. The process includes the step of contacting a chiral alpha-amino carbonyl compound and hydrogen, in the presence of a non-racemic hydrogenation catalyst, at a temperature, pressure and for a length of time sufficient to produce the non-racemic aminoalcohol. In a preferred embodiment, the process can be described by the reaction scheme: 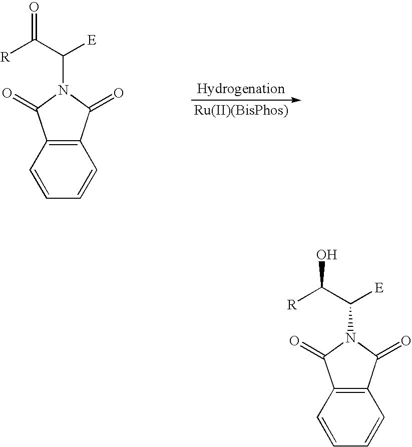
where R is hydrogen, alkyl, substituted alkyl, aryl, substituted aryl or hetereoaryl group; and E can be hydrogen, COOR, CONHR, CONR₂, COOH, COR, CN, NO₂, alkyl, substituted alkyl, aryl, substituted aryl or hetereoaryl group.

This application claims priority from U.S. Provisional Application Ser. No. 60/536,637, filed Jan. 15, 2004.

### BACKGROUND OF THE INVENTION

1. Field of the Invention

The present invention relates to development of new methods for preparation of chiral aminoalcohols through asymmetric hydrogenation of alpha-amino carbonyl compounds with a variety of groups linked to the amines. More particularly, high activities, enantioselectivities and diasetereoselectivities hydrogenation can be obtained when alpha phthalimide carbonyl compounds are used as substrates.

2. Description of the Prior Art

As the responsible function group of biologically active molecules as well as a useful building block, aminoalcohol is an extremely important unit in organic synthetic chemistry. How to construct the structure motif attracts extensive efforts of organic chemist. Developing highly enantioselective method to prepare aminoalcohol with efficiency remains one of the major challenges. No doubt, asymmetric hydrogenation is the most powerful method to construct one or two chiral centers.

The elegant asymmetric hydrogenation of ketones is generally regarded as being the most successful method to form chiral alcohols (Noyori, R., Angew. Chem., Int. Ed., 2002, 41, 2008). However, there are few successes in ketone hydrogenation when an α-NH₂ group exists.

Some aminoketone substrates have been used for asymmetric hydrogenation (Noyori, R.; Ikeda, T.; Ohkuma, T.; Widhalm, M.; Kitamura, M.; Takaya, H.; Akutagawa, S.; Sayo, J. Amer. Chem. Soc., 1989, 111, 9134-9135). In this hydrogenation, syn-aminoalcohol products have been observed in the dynamic hydrogenation.

Recently, Noyori at al. applied RuCl₂(bisphosphine)(1,2-diamine) complexes in the asymmetric hydrogenation of amino ketones in the presence of strong base, which are efficient catalysts for unfunctionalized ketones (Ohkuma, T.; Koizumi, M.; Muniz, K.; Hilt, G.; Kabuto, C.; Noyori, R., J. Am. Chem. Soc. 2000, 122, 6510-6511, Katayama, Eiji; Sato, Daisuke; Ooka, Hirohito; Inoue, Tsutomu, Int. Appl., 2000, WO 2000041997).

In this invention, we describe highly enantioselective asymmetric hydrogenation of alpha-amino carbonyl compounds, such as, α-phthalimide ketones, to form α-phthalimide alcohols, which are masked α-primary aminoalcohols.

High anti selectivities have been observed in the dynamic hydrogenations in the synthesis of aminoalcohols.

### SUMMARY OF THE INVENTION

The present invention provides a process for preparing a non-racemic aminoalcohol. The process includes the step of contacting a chiral alpha-amino carbonyl compound and hydrogen, in the presence of a non-racemic hydrogenation catalyst, at a temperature, pressure and for a length of time sufficient to produce the non-racemic aminoalcohol.

The chiral alpha-amino carbonyl compound is represented by formula: 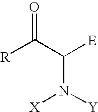
and the non-racemic aminoalcohol is represented by formula: 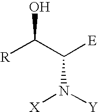
wherein R is selected from hydrogen, alkyl, substituted alkyl, aryl, substituted aryl and hetereoaryl group;
E is selected from hydrogen, COOR, CONHR, CONR₂, COOH, COR, CN, NO₂, alkyl, substituted alkyl, aryl, substituted aryl and hetereoaryl group; and
each X and Y is independently selected from hydrogen, R, OH, NH₂, OCOR, NHCOR, POR₂, COR, COOR, CONHR and CONR₂; or wherein X and Y together with the nitrogen atom N, form a cyclic imide group.

### DETAILED DESCRIPTION OF THE INVENTION

In a preferred embodiment, the present process is carried out via a reaction scheme shown below:

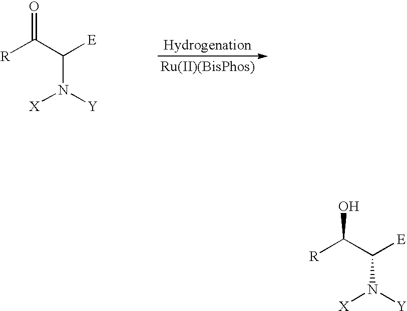

In a preferred embodiment of this invention, R is a hydrogen, an alkyl, substituted alkyl, aryl, substituted aryl, hetereoaryl group; E is a hydrogen, COOR, CONHR, CONR₂, COOH, COR, CN, NO₂, alkyl, substituted alkyl, aryl, substituted aryl, and hetereoaryl group; X, Y, independently, can be hydrogen, R, OH, NH₂, OCOR, NHCOR, POR₂, COR, COOR, CONHR, CONR₂; or X, Y together with the nitrogen atom N is a cyclic imide, such as, phthalimide.

Preferably, the cyclic imide can be phthalimide, dihydrophthalimide, tetrahydrophthalimide, succinimide, alkylsuccinimide, maleimide, or alkylmaleimide and the alpha-amino carbonyl compound can be an alpha-amino ketone.

The non-racemic hydrogenation catalyst can be formed from a non-racemic ligand and a transition metal, a salt thereof, or complex thereof. The preferred transition metals include: Pt, Pd, Rh, Ru, Ir, Cu, Ni, Mo, Ti, V, Re and Mn, the most preferred transition metals being selected from Pd, Rh, Ru and Ir.

The suitable transition metal salts and complexes include PtCl₂; Pd₂(DBA)₃; Pd(OAc)₂; PdCl₂(RCN)₂; (Pd(allyl)Cl)₂; (Rh(COD)Cl)₂; (Rh(COD)₂)X; Rh(acac)(CO)₂; Rh(ethylene)₂(acac); Rh(CO)₂Cl₂; Ru(RCOO)₂(diphosphine); Ru(methylallyl)2(diphosphine); Ru(aryl group)X₂(diphosphine); RuCl₂(COD); (Rh(COD)₂)X; RuX₂(diphosphine); RuCl₂(═CHR)(PR′₃)₂; Ru(ArH)Cl₂; Ru(COD)(methylallyl)₂; Ru(arene)X₂(bisphos); Ru(RCOO)₂(bisphos); Ru(CF₃COO)₂(bisphos); Ru(methallyl)₂(bisphos); RuX₂(cymen)(bisphos); RuHX(bisphos); [Ru₂X₅(bisphos)₂]NH₂Me₂; [Ru₂X₅(bisphos)₂]NH₂Et₂; (Ir(COD)₂Cl)₂; (Ir(COD)₂)X; Cu(OTf); Cu(OTf)₂; Cu(Ar)X; CuX; NiX₂; Ni(COD)₂; MoO₂(acac)₂; Ti(OiPr)₄; VO(acac)₂; MeReO₃; MnX₂ and Mn(acac)₂; wherein each R and R′ can independently be alkyl or aryl; Ar is an aryl group; and X is a counteranion.

The counteranion X can be halogen, BF₄, B(Ar)₄ wherein Ar is 3,5-di-trifluoromethyl-1-phenyl, ClO₄, SbF₆, CF₃SO₃, RCOO or a mixture thereof.

The catalyst can be prepared in situ or it can be obtained as an isolated compound.

The preferred Ru(II) catalysts include Ru(arene)X₂(bisphos), Ru(RCOO)₂(bisphos), Ru(CF₃COO)₂(bisphos), Ru(methallyl)₂(bisphos), RuX₂(cymen)(bisphos), RuHX(bisphos), [Ru₂X₅(bisphos)₂]NH₂Me₂, [Ru₂X₅(bisphos)₂]NH₂Et₂. X is Cl, Br, or I.

Typically, the non-racemic hydrogenation catalyst is a non-racemic mixture of enantiomers. Preferably, the non-racemic hydrogenation catalyst is one of the enantiomers, having an optical purity of at least 95% ee, more preferably, at least 98% ee. However, non-racemic hydrogenation catalysts having an optical purity of less than 95% ee, but at least 85% ee, or even at least 75% ee, can also be used.

The preferred bisphosphine ligands, also referred to herein as “diphosphine” ligands, that are used to prepare the catalysts according to the present invention, include BINAP, substituted BINAP, MeO-BIPHEP, TunePhos, SEGPhos, H₈BINAP, Cl- or MeO-BIPHEP (i,e, chloro or methoxy disubstituted BIPHEP), BIPFUP, BITIAP, BITIOP, SynPhos, P-Phos, O-BIPEP, DuPhos, Ferrotane, JosiPhos, WalPhos, MandyPhos, TaniaPhos, JafaPhos, f-KetalPhos, f-Binaphane, BPE, Rophos, ButiPhane, PennPhos, MalPhos, KetalPhos, Binaphane, BICP, DeguPhos, DIOP*, Dipamp, TangPhos, Binapine and other chiral bisphosphorous ligands.

These and other suitable ligands are described in detail in a review article entitled “New Chiral Phosphorus Ligands for Enantioselective Hydrogenation” by W. Tang and X. Zhang, Chem. Reviews, vol. 103, pages 3029-3069 (2003), the contents of which are incorporated herein by reference as fully set forth.

Some specific chiral phosphines are illustrated in scheme A to Scheme I that follow.

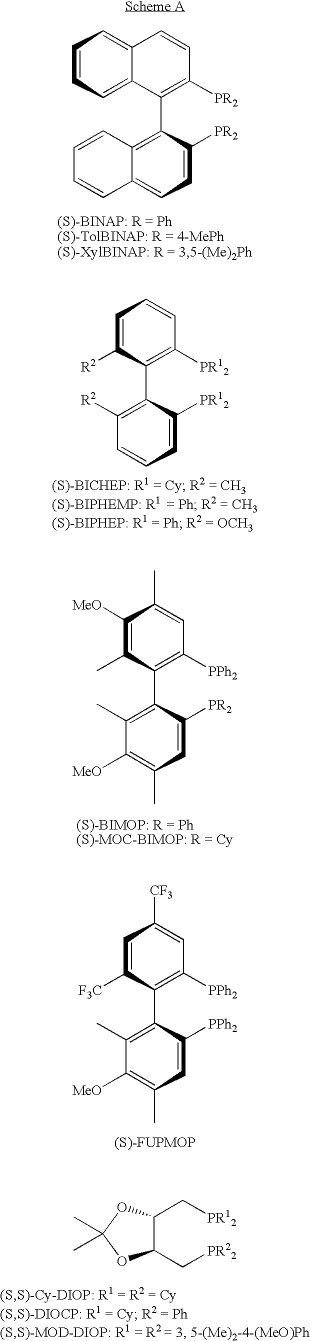

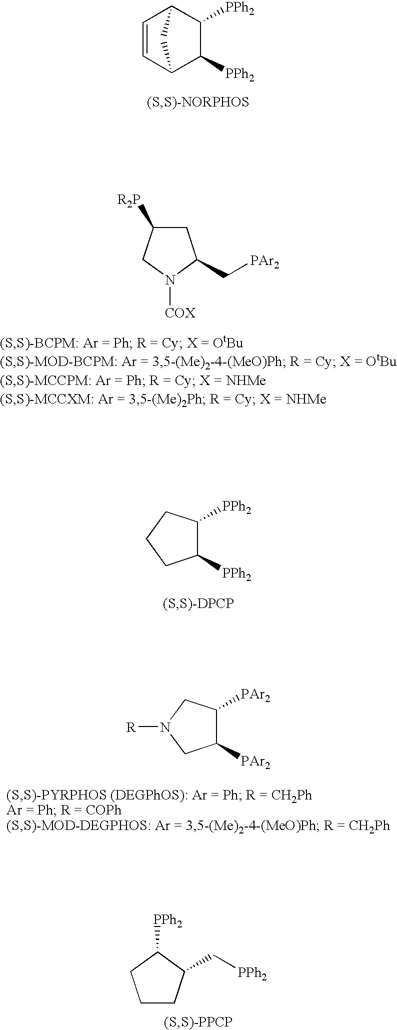

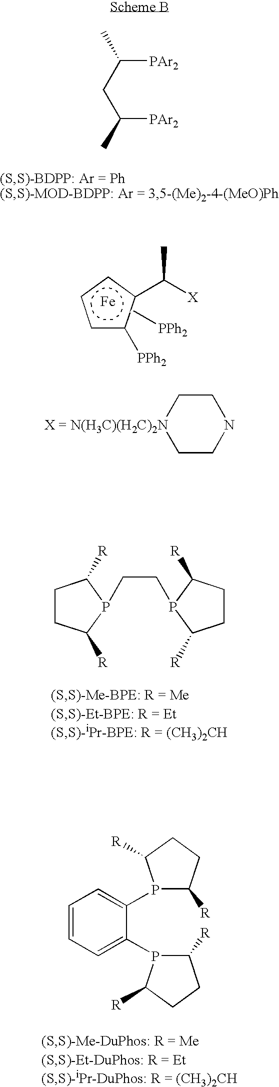

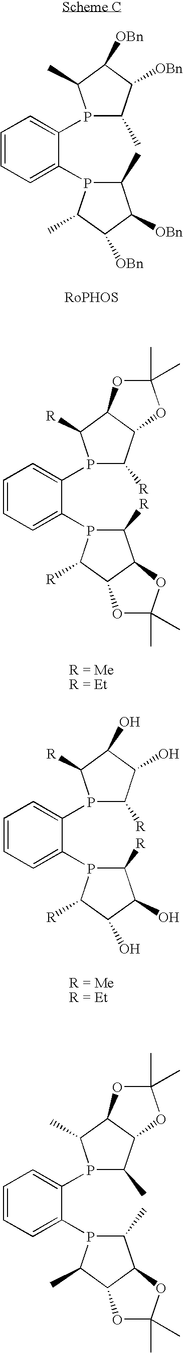

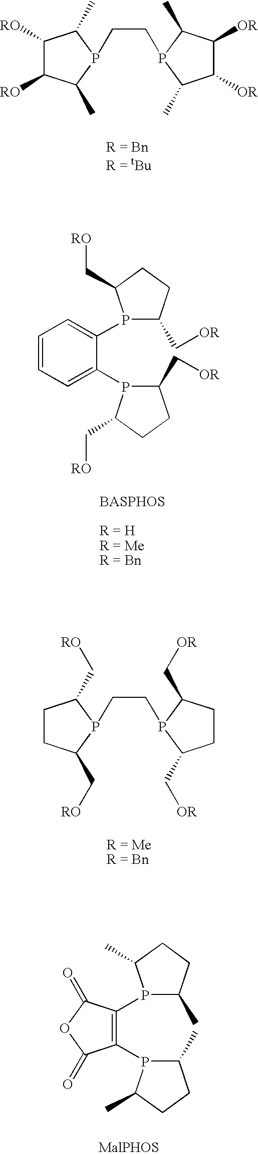

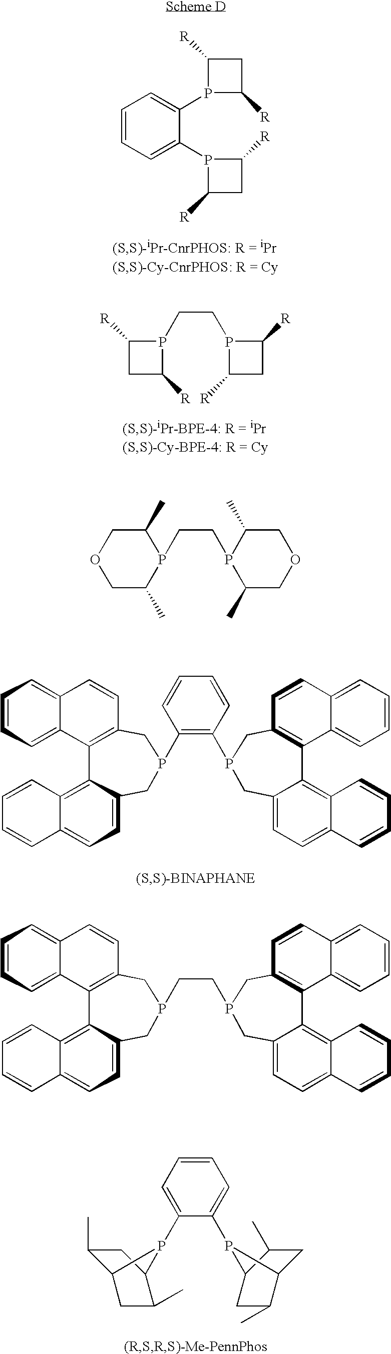

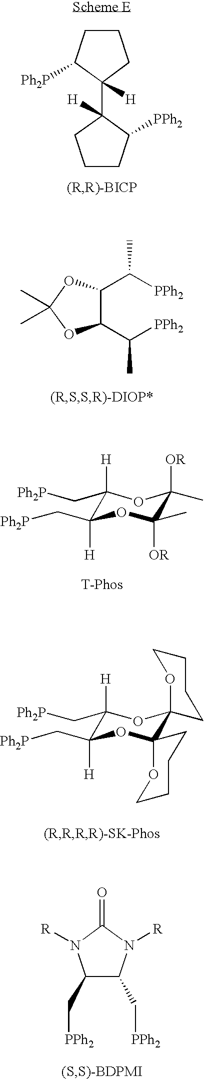

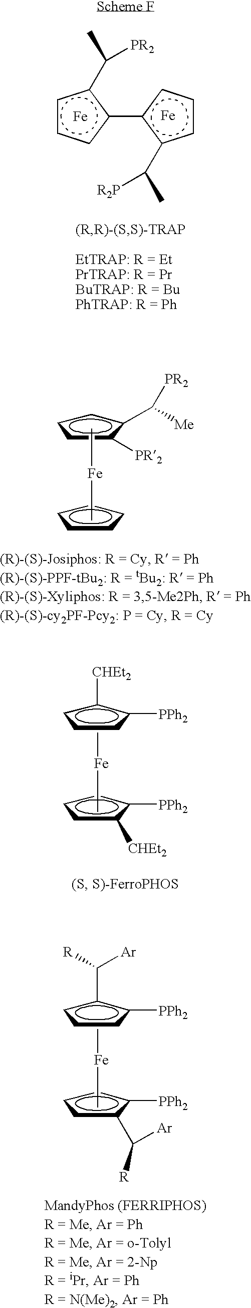

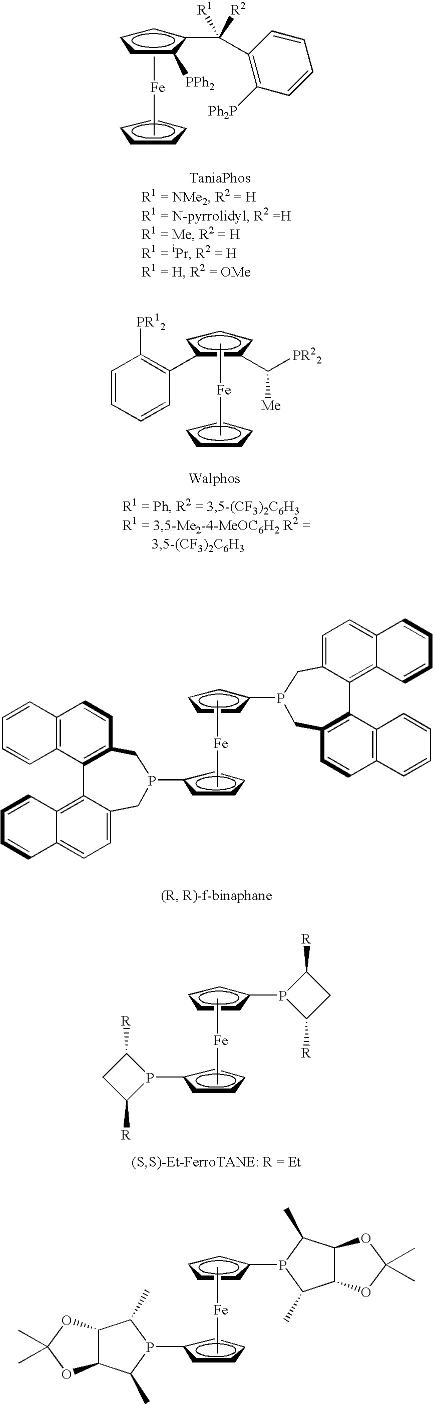

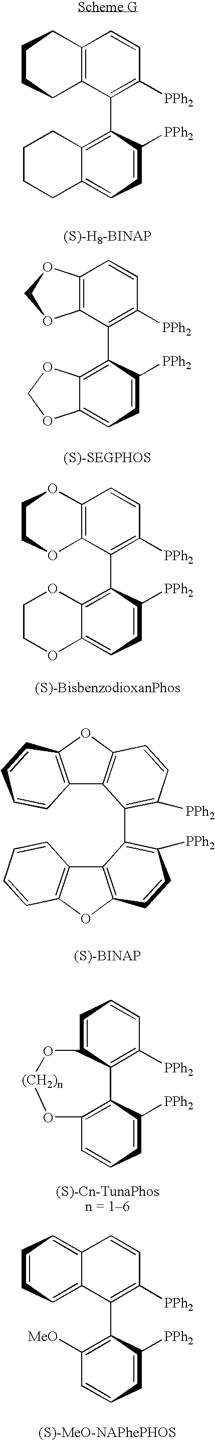

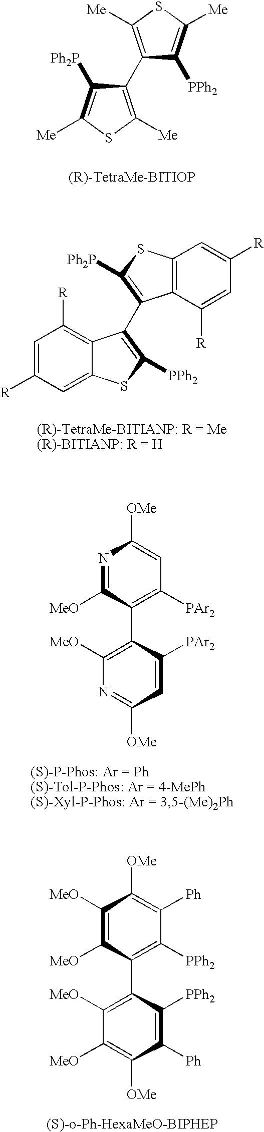

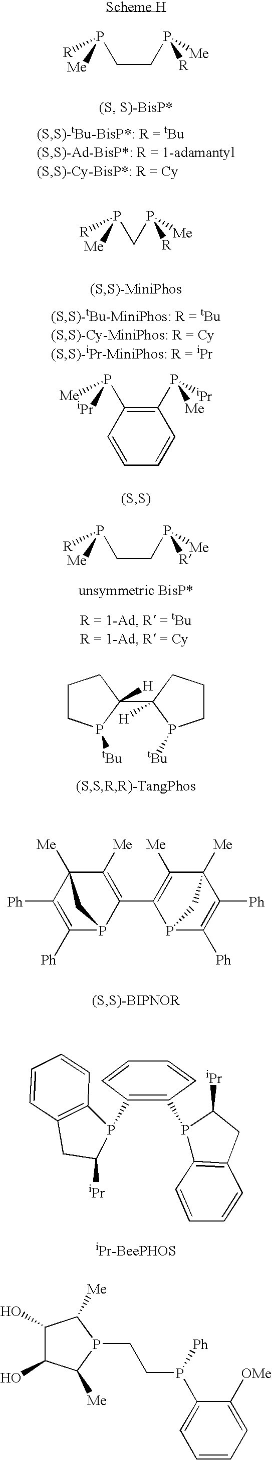

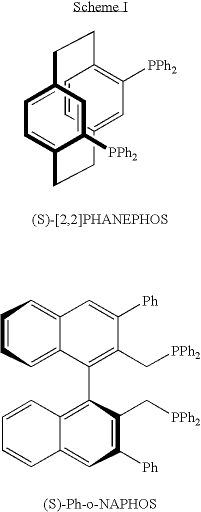

The non-racemic aminoalcohols that can be prepared by the process of the present invention include compounds represented by the following formulas:

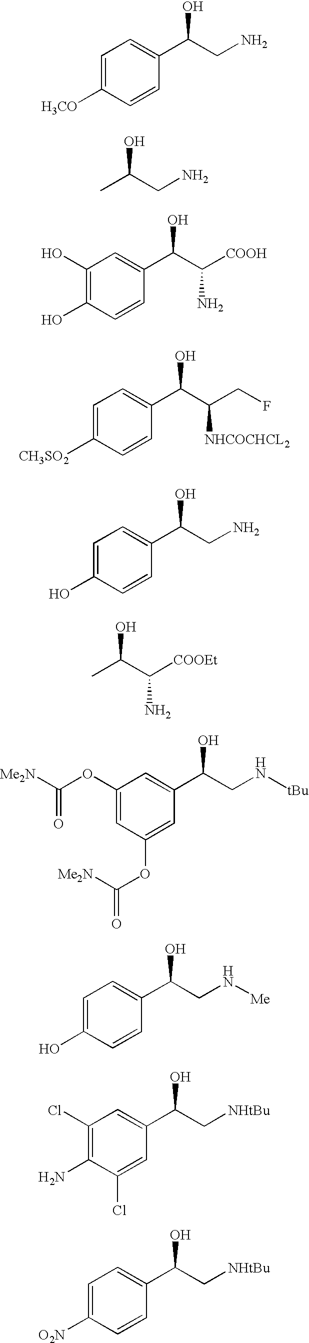

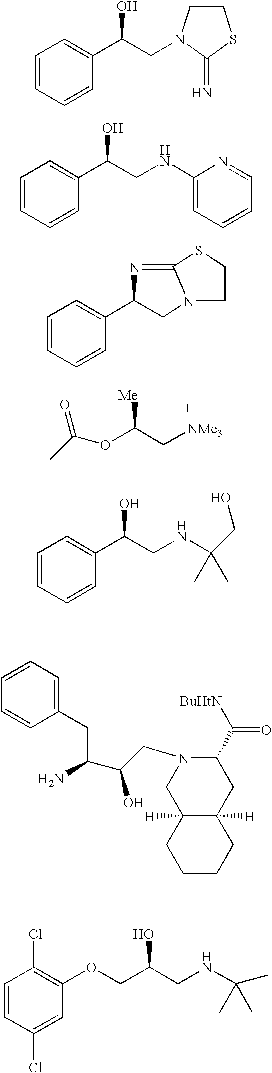

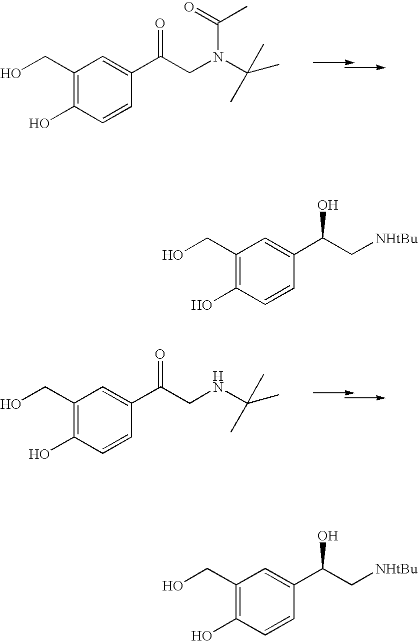

The latter non-racemic aminoalcohol represented by the formula: 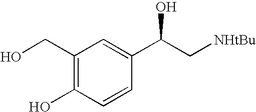
can be formed from one or more of the compounds represented by the formula:

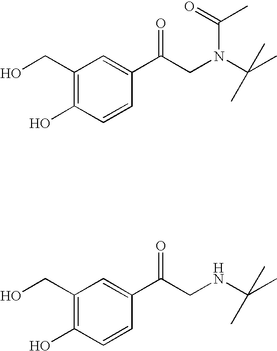

Thus, either the free amine or an acylated derivative thereof, such as, the acetylated derivative shown above, or even a mixture of the free amine and an acylated derivative of the free amine can be used to prepare the aminoalcohol shown above. This is possible because, under the reaction conditions used, the acyl group is easily removed to produce the free aminoalcohol.

Preferably, the non-racemic aminoalcohol has an optical purity of at least 95% ee, more preferably, at least 98% ee. However, non-racemic aminoalcohol having an optical purity of less than 95% ee, but at least 85% ee, or even at least 75% ee, are also very useful in production of pharmaceutical, agricultural, and other types of commercially important compounds.

In a preferred example of the present process, the reaction is carried out as shown below: 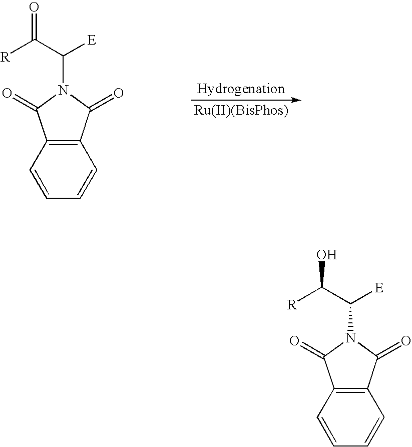
wherein R is selected from hydrogen, alkyl, substituted alkyl, aryl, substituted aryl and hetereoaryl group; and
E is selected from hydrogen, COOR, CONHR, CONR₂, COOH, COR, CN, NO₂, alkyl, substituted alkyl, aryl, substituted aryl and hetereoaryl group.

Typically, the step of contacting the chiral alpha-amino carbonyl compound and hydrogen in the presence of a non-racemic hydrogenation catalyst is carried out at a temperature, pressure and for a length of time sufficient to produce the non-racemic aminoalcohol.

The conditions that are sufficient to produce the non-racemic aminoalcohols are described in detail in the Examples below.

Hydrogenation N-phenacyl-phthalimide Using Different Catalysts

The results of these hydrogenations are shown in Table 1.

Firstly, high activity and mild condition when the [Rh(NBD)(Tangphos)]SbF₆ 1 was chosen as catalyst precursor. Unfortunately, all the efforts to improve the ee value met with limited success when catalyst 1 was used.

Catalyst 2 showed even lower reactivity and no satisfied enantioselectivity. Catalyst 3d was inactive at room temperature and 30 psi hydrogen pressure.

An unexpected result was observed when we tried to improve ee using methanol at 80° C. and 1500 psi hydrogen pressure, in which the ee value jumped to 90.1% when 3d was used as catalyst (Table 1, entry 6).

During the process of condition optimization, we observed an interesting solvent-depended phenomenon. Only using EtOH as the solvent, the hydrogenation takes place smoothly in 95.1% ee and 100% conversion (Table 1, entry 11).

When other solvents such as toluene, CH₂Cl₂, EtOAc, ClCH₂CH₂Cl and THF (Table 1, entries 7-10) were employed, the reaction turns out very slow; even using similar solvent such as methanol, isopropanol, and n-propanol, the low reactivity was observed (Table 1, entries 6, 12).

TABLE 1
Asymmetric Hydrogenation of N-Phenacyl-phthalimid.

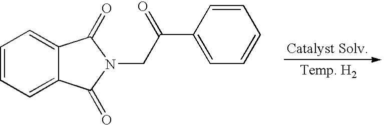

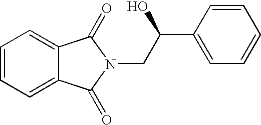

| | | Temp. | | | | |
|---|---|---|---|---|---|---|
| Entry | Slov. | (° C.) | H₂ (psi) | Catalyst | ee(%) | Conv. (%) |
| 1 | CH₂Cl₂ | rt | 30 | 1 | 17.0 | 100 |
| 2 | CH₂Cl₂ | rt | 30 | 2 | 27.0 | 35 |
| 3 | CH₂Cl₂ | rt | 30 | 3d | / | 0 |
| 4 | MeOH | 80 | 1500 | 1 | 10.0 | 100 |
| 5 | MeOH | 80 | 1500 | 2 | 29.0 | 100 |
| 6 | MeOH | 80 | 1500 | 3d | 90.1 | 29 |
| 7 | CH₂Cl₂ | 80 | 1500 | 3d | 0 | 0 |
| 6 | THF | 80 | 1500 | 3d | 0 | 25 |
| 8 | Toluene | 80 | 1500 | 3d | 50.2 | 11 |
| 9 | ClCH₂CH₂Cl | 80 | 1500 | 3d | 0 | 12 |
| 10 | EtOAc | 80 | 1500 | 3d | 17.3 | 9 |
| 11 | EtOH | 80 | 1500 | 3d | 95.1 | 100 |
| 12 | IPA | 80 | 1500 | 3d | 33.7 | 70 |
| 13 | EtOH | 80 | 1500 | 3a | 91.3 | 70 |
| 14 | EtOH | 80 | 1500 | 3b | 90.3 | 72 |

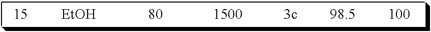

| | | | | | | |
|---|---|---|---|---|---|---|
| 16 | EtOH | 80 | 1500 | 3e | 95.3 | 100 |
| 17 | EtOH | 80 | 1500 | 3f | 90.7 | 100 |
| 18 | EtOH | 80 | 1500 | 4 | 94.3 | 100 |
| 19 | EtOH | 80 | 1500 | 5 | 96.1 | 100 |

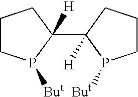

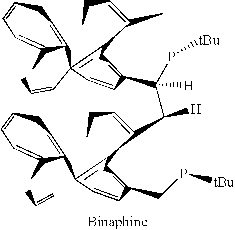

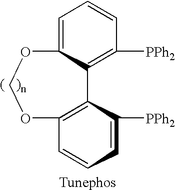

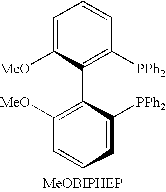

| | | | | |
|---|---|---|---|---|
| Catalysts: | [Rh(NBD)(TangPhos)]SbF₆ | 1 | Ru-(MeOBIPHEP) | 4 |
| | [Rh(NBD)(BINAPINE)]SbF₆ | 2 | Ru-(BINAP) | 5 |

| | |
|---|---|
| Ru-(TunePhos) | n = 1 3a; n = 2 3b; |
| | n = 3 3c; n = 4 3d; |
| | n = 5 3e; n = 6 3f; |

Superior results were obtained using C₃-Tunephos as the ligand and [NH₂Me₂][{RuCl(C3-tunephos)}(μ-Cl)₃] as the catalyst (Zhang, Z.; Quian, H.; Longmire, J.; and Zhang, X. J., Org. Chem., vol. 65, page 6223 (2000), in which 98.5% ee and 100% conversation was obtained using Catalyst 3c. Using MeO-BIPHEP and BINAP as the ligands under the same condition, the ee values were 94.3% and 96.1%, respectively.

TABLE 2
Asymmetric Hydrogenation of α-Phthalimide Ketone^{a}

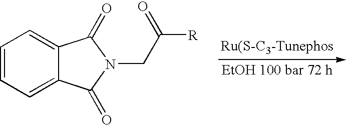

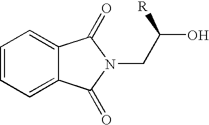

| entry | R | Temp. (° C.) | Conv. (%) | ee (%)^{b} |
|---|---|---|---|---|
| 1 | P-MeOC₆H₅ | 80 | 100 | 95.3 |
| 2 | P-MeOC₆H₅ | 60 | 60 | 98.5 |
| 3 | P-MeC₆H₅ | 80 | 100 | >99.0 |
| 4 | P-FC₆H₅ | 60 | 100 | >99.0 |
| 5 | P-ClC₆H₅ | 80 | 100 | 92.3 |
| 6 | P-ClC₆H₅ | 60 | 30 | 94.0 |
| 7 | P-BrC₆H₅ | 80 | 100 | >99.0 |
| 8 | m-MeOC₆H₅ | 80 | 100 | >99.0 |
| 9 | o-MeOC₆H₅ | 60 | 100 | >99.0 |
| 10 | Me | 60 | 100 | >99.0 |
| 11 | Et | 80 | 100 | >99.0 |

^{a}The reaction was carried out with 2 mol% Ru catalyst.
^{b}The ee values were detected via HPLC.

The scope of the suitable substrates is apparent from Table 2. Both electron-deficient and electron-rich aryl ketones can be reduced in high enantioselectivity. The position of substituents was also widely compatible for the high enantioselectivity. No matter which of the o-, m- or p-methoxy aryl ketones was hydrogenated, the ee values were always higher than 98.5%.

In addition, the compatibility of functional groups was also examined. It was found that an aryl fluoride, chloride and even aryl bromide can be present in the substrates without any deleterious effect on the reaction.

Further, alkyl ketones and even simple methyl ketones worked well and gave high enantioselectivity (Table 2, entries 10 and 11). Thus, clearly, other functional groups can be used advantageously to extend the synthetic applications of the present invention.

Scheme 1 shows the synthetic details of these reactions.

The starting materials can be obtained in really economical and large scale starts from chloroacetone and phthalimide in almost quantitative yield.

Following hydrogenation with 10,000 TON without further optimization of the reaction conditions, the desired product was obtained in over 99% ee.

The step of hydrolyzing the phthalimide to provide the (S)-(+)-1-amino-2-propanol was conducted in ethanol at reflux in the presence of NH₂NH₂.

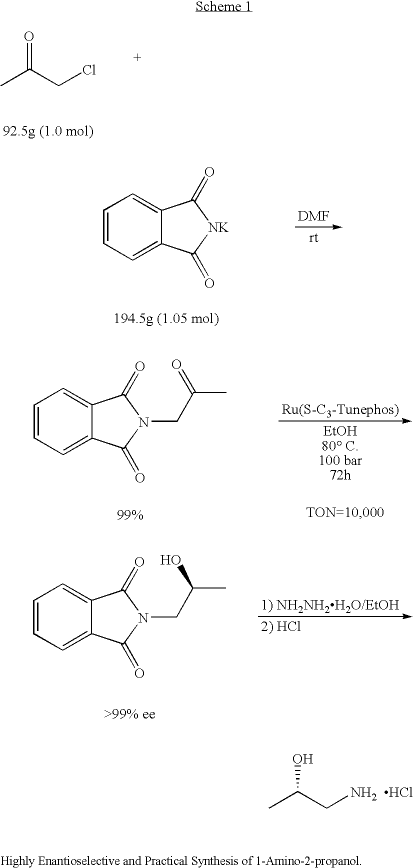

Scheme 2 illustrates the hydrogenation of α-phthalimide ketones to produce optically pure aminoalcohols in excellent enantioselectivity. An example of the use of this reaction is the synthesis of threonine by dynamic kinetic resolution (Scheme 2).

Using catalyst 3c, the allo-threonine was obtained in over 99% ee and >97:3 dr. Compared with Noyori's system (Noyori, R.; Ikeda, T.; Ohkuma, T.; Widhalm, M.; Kitamura, M.; Takaya, H.; Akutagawa, S.; Sayo, J. Am. Chem. Soc., 1989, 111, 9134-9135), not only the catalyst was different, but the syn/anti selectivity was totally reversed. We obtained over 97:3 ratio of antilsyn selectivity.

Thus, using the (R-C₃-Tunephos and S-C₃-tunephos) catalyst, both (2R 3R)-(−)-allo- and (2S,3S)-(+)-allo-threonine are obtained in high optical purity, in which the allo-threonines are the more expensive isomers compared with threonine.

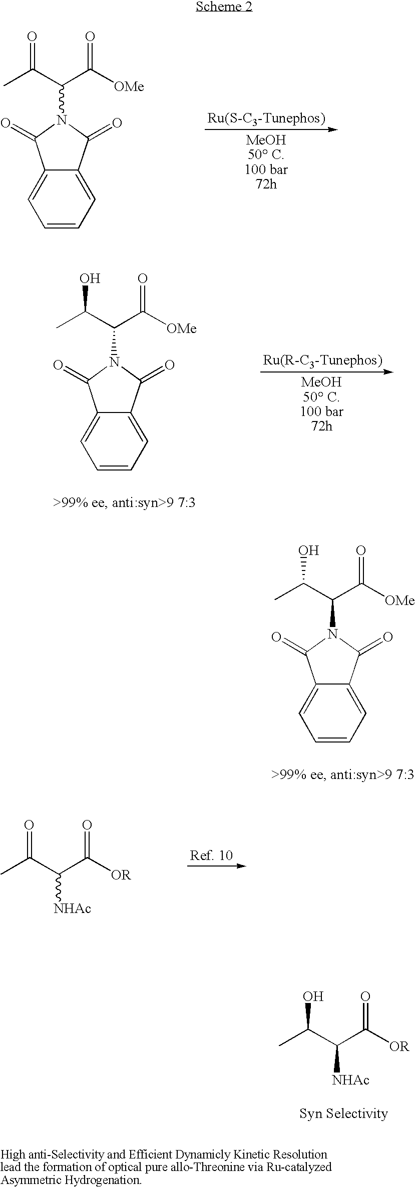

Thus, it can be seen from the above, that the process according to the present invention provides an efficient method of synthesis of optically pure aminoalcohols, which are an important class of compounds having a variety of uses in synthetic chemistry, medicinal chemistry, and bioorganic chemistry.

General Methods:

All reactions were carried out under inert atmosphere using standard Schlenk techniques. Column chromatography was performed on EM silica gel 60 (200-400 mesh). ¹H NMR and ¹³C NMR spectra were recorded on Bruker DPX-300, DRX-300, DRX-400 and AMX-360 spectrophotometers.

General Procedure for the Syntheses of Phthalimide Ketones:

In a dried flask, to a solution of α-bromide or (chloride)-ketone (10 mmol) in DMF (10 mL) was added 110 mol % potassium phthalimide with stirring (the reaction can be carried out in the air without special handling; potassium phthalimide was not completely dissolved in the DMF). The reaction was run at room temperature and monitored by TLC. After the reaction was complete, the reaction mixture was poured into water (250 mL). The desired products yield was collected by filtration. Further purification can be obtained via recrystalyzation from ethanol or isopropanol.

General Procedure: Asymmetric Hydrogenation of Phthalimide Ketones

To the solution of [NH₂Me₂][{RuCl(bisphos)}(μ-Cl)₃] was added the substrate, this solution was then transferred into an autoclave. The hydrogenation was performed at a given temperature under pressure of H₂. The bisphos used in this study include TunePhos (Tunaphos), BINAP, Meo-BIPHEP and other ligands.

After carefully releasing the hydrogen, the reaction mixture was evaporated. The residue was re-dissolved with ethyl acetate, which was subsequently passed through a short silica gel plug to remove the catalyst.

The resulting solution was directly used for chiral GC or HPLC to measure the enantiomeric excesses.

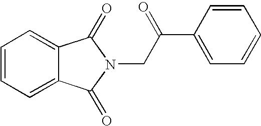

¹H NMR (400 MHz, CDCl₃) δ 7.97 (d, J=8.0 Hz, 2H), 7.86-7.83 (m, 2H), 7.72-7.69 (m, 2H), 7.61-7.56 (m, 1H), 7.47 (t, J=7.8 Hz, 2H), 5.10 (s, 2H); ¹³C NMR (100 MHz, CDCl₃) δ 191.39, 168.28, 134.78, 134.54, 134.44, 132.61, 129.29, 128.54, 123.92, 44.60; MS (APCI) m/z: [M⁺30 1], 266.1;

HRMS (APCI), Caclt'd for C₁₆H₁₂NO₃ [M+1]: 266.0812, found: 266.0819.

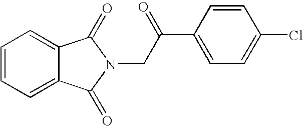

¹H NMR (400 MHz, CDCl₃) δ 7.99-7.87 (m, 4H), 7.75-7.40 (m, 2H), 7.47 (d, J=8.0 Hz, 2H), 5.07 (s, 2H); ¹³C NMR (100 MHz, CDCl₃) δ 190.35, 168.22, 141.01, 134.61, 133.11, 132.56, 129.94, 129.67, 124.01, 44.48; MS (APCI) m/z: [M⁺+1], 300.0; HRMS (APCI), Caclt'd for C₁₆H₁₁NClO₃[M+1]: 300.0422, found: 300.0433.

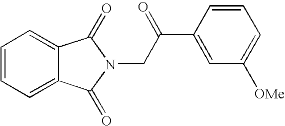

¹H NMR (300 MHz, CDCl₃) δ 7.89-7.86 (m, 2H), 7.74-7.71 (m, 2H), 7.57 (d, J=7.6 Hz, 1H), 7.48 (s, 1H), 7.40 (t, J=8.0 Hz, 1H), 7.15 (dd, J=2.6, 8.3 Hz, 1H), 5.09 (s, 2H), 3.83 (s, 3H); ¹³C NMR (75 MHz, CDCl₃) δ 191.27, 168.28, 160.36, 136.08, 134.54, 132.62, 130.30, 123.95, 121.03, 120.98, 112.75, 55.89, 44.70; MS (APCI) m/z: [M⁺+1], 296.1; HRMS (APCI), Caclt'd for C₁₇H₁₄NO₄ [M+H]: 296.0917, found: 296.0916.

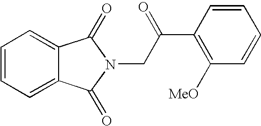

¹H NMR (300 MHz, CDCl₃) δ 7.93-7.85 (m, 3H), 7.72-7.70 (m, 2H), 7.52 (dt, J=1.8, 7.4 Hz, 1H), 7.00 (d, J=7.4 Hz, 2H), 5.06 (s, 2H), 3.98 (s, 3H); ¹³C NMR (75 MHz, CDCl₃) δ 192.22, 168.57, 160.16, 135.57, 134.38, 132.73, 131.77, 124.74, 123.83, 121.38, 111.99, 56.02, 49.06; MS (APCI) m/z: [M⁺30 1], 296.1; HRMS (APCI), Caclt'd for C₁₇H₁₄NO₄[M+H]: 296.0917, found: 296.0918.

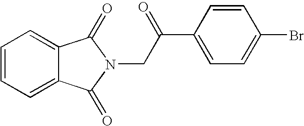

¹H NMR (300 MHz, CDCl₃) δ 7.89-7.84 (m, 4H), 7.75-7.72 (m, 2H), 7.66-7.63 (m, 2H), 5.06 (s, 2H); ¹³C NMR (100 MHz, CDCl₃) δ 190.53, 168.21, 134.61, 133.51, 132.68, 132.56, 129.99, 129.77, 124.02, 44.45; MS (APCI) m/z: [M⁺30 1], 344.0; HRMS (APCI), Caclt'd for C₁₆H₁₁NBrO₃[M+1]: 343.9917, found: 343.9920.

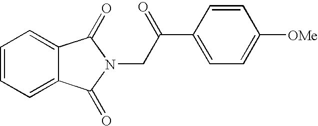

¹H NMR (300 MHz, CDCl₃) δ 7.94 (d, J=8.9 Hz, 2H), 7.85-7.80 (m, 2H), 7.72-7.67 (m, 2H), 6.92 (d, J=8.9 Hz, 2H), 5.05 (s, 2H), 3.83 (s, 3H); ¹³C NMR (75 MHz, CDCl₃) δ 189.76, 168.36, 164.54, 134.48, 132.63, 130.84, 127.79, 123.87, 114.46, 55.94, 44.27; MS (APCI) m/z: [M⁺30 1], 296.1; HRMS (APCI), Caclt'd for C₁₇H₁₄NO₄ [M+1]: 296.0917, found: 296.0920.

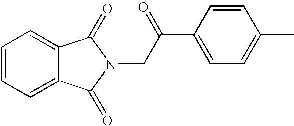

¹H NMR (400 MHz, CDCl₃) δ 7.88-7.83 (m, 4H), 7.73-7.69 (m, 2H), 7.27 (d, J=7.9 Hz, 2H), 5.08 (s, 2H), 2.40 (s, 3H); ¹³C NMR (100 MHz, CDCl₃) δ 190.92, 168.33, 145.41, 134.50, 132.65, 132.33, 130.05, 129.88, 128.60, 123.91, 44.50, 22.17.

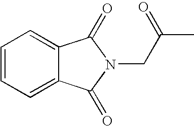

¹H NMR (400 MHz, CDCl₃) δ 7.83-7.77 (m, 2H), 7.70-7.66 (m, 2H), 4.46 (s, 2H), 2.22 (s, 3H); ¹³C NMR (100 MHz, CDCl₃) δ 200.14, 168.01, 134.57, 132.41, 123.89, 47.48, 27.39; MS (APCI) m/z: [M⁺30 1], 204.1;

HRMS (APCI), Caclt'd for C₁₁H₁₀NO₃[M+1]: 204.0655, found: 204.0670.

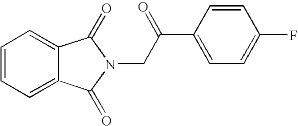

¹H NMR (400 MHz, CDCl₃) δ 8.02-7.99 (m, 2H), 7.87-7.85 (m, 2H), 7.73-7.71 (m, 2H), 7.15 (t, J=8.5 Hz, 2H), 5.07 (s, 2H); ¹³C NMR (100 MHz, CDCl₃) δ 189.89, 168.23, 134.58, 132.56, 131.24, 123.96, 116.73, 116.52, 116.35, 44.43; MS (APCI) m/z: [M⁺30 1], 284.1; HRMS (APCI), Caclt'd for C₁₆H₁₁NFO₃[M+NH4]: 284.0718, found: 284.0707.

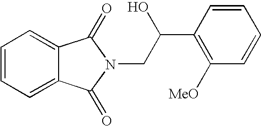

[α]=+40.5 C=1.0 in CHCl₃ (from (S)-C₃-Tunephos)

¹H NMR (300 MHz, CDCl₃) δ 7.82-7.79 (m, 2H), 7.70-7.67 (m, 2H), 7.33 (dd, J=1.5, 7.4 Hz, 1H), 7.24 (dt, J=1.6, 8.0 Hz, 1H), 6.93-6.86 (m, 2H), 5.13-5.11 (m, 1H), 4.15 (dd, J=8.3, 14.0 Hz, 1H), 3.97 (dd, J=4.1, 14.0 Hz, 1H), 3.88 (s, 3H), 3.36 (br, 1H); ¹³C NMR (75 MHz, CDCl₃) δ 166.97, 155.02, 132.17, 130.23, 127.29, 126.85, 125.78, 121.50, 119.04, 108.79, 68.66, 53.57, 42.07; MS (APCI) m/z: [M-OH], 280.1; HRMS (APCI), Caclt'd for C₁₇H₁₄NO₃[M-OH]: 280.0968, found: 280.0969.

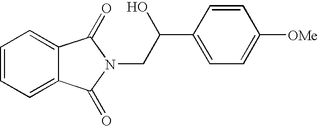

[α]=−18.8 C=1.0 in CHCl₃ (from (R)-C₃-Tunephos)

¹H NMR (300 MHz, CDCl₃) δ 8.08-7.99 (m, 2H), 7.82-7.80 (m, 2H), 7.30-7.28 (m, 2H), 6.86-6.83 (m, 2H), 4.66-4.62 (m, 1H), 3.99 (dd, J=8.6, 14.0 Hz, 1H), 3.77 (s, 3H), 3.73 (dd, J=5.5, 14.0 Hz, 1H); ¹³C NMR (75 MHz, CDCl₃) δ 168.58, 159.89, 134.25, 132.44, 131.82, 128.58, 123.65, 114.28, 64.64, 55.63, 44.44; MS (APCI) m/z: [M⁺-OH], 280.1; HRMS (APCI), Caclt'd for C₁₇H₁₄NO₃[M-OH]: 280.0968, found: 280.0965.

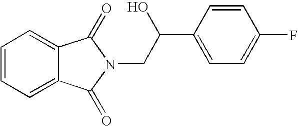

[α]=+18.3 C=1.0 in CHCl₃ (from (S)-C₃-Tunephos)

¹H NMR (300 MHz, CDCl₃) δ 7.84-7.81 (m, 2H), 7.72-7.69 (m, 2H), 7.42-7.37 (m, 2H), 7.06-6.98 (m, 2H), 5.05-5.02 (m, 1H), 4.10-3.86 (m, 2H), 2.99 (br, 1H); ¹³C NMR (75 MHz, CDCl₃) δ 169.14, 137.19, 134.62, 132.17, 128.05, 127.94, 123.92, 116.02, 115.73, 72.44, 46.12; MS (APCI) m/z: [M-OH], 268.1; HRMS (APCI), Caclt'd for C₁₆H₁₁NFO₂ [M-OH]: 268.0768, found: 268.0749.

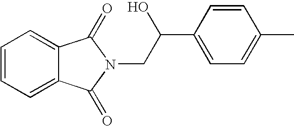

[α]=−23.8 C=1.0 in CHCl₃ (from (R)-C₃-Tunephos)

¹H NMR (300 MHz, CDCl₃) δ 7.77-7.73 (m, 2H), 7.67-7.63 (m, 2H), 7.25 (d, J=8.0 Hz, 2H), 7.09 (d, J=7.8 Hz, 2H), 4.97-4.93 (m, 1H), 4.04-3.71 (m, 2H), 2.26 (s, 3H); ¹³C NMR (75 MHz, CDCl₃) δ 169.15, 138.49, 138.22, 134.50, 132.29, 129.67, 126.22, 123.84, 72.81, 46.09, 21.55; MS (APCI) m/z: [M⁺-OH], 264.1; HRMS (APCI), Caclt'd for C₁₇H₁₄NO₂[M-OH]: 264.1019, found: 264.1039.

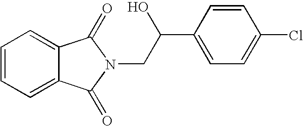

[α]=−11.3 C=1.0 in CHCl₃ (from (R)-C₃-Tunephos)

¹H NMR (300 MHz, CDCl₃) δ 7.78-7.76 (m, 2H), 7.67-7.65 (m, 2H), 7.33-7.19 (m, 4H), 4.99-4.96 (m, 1H), 4.05-3.81 (m, 2H), 3.02 (br, 1H); ¹³C NMR (75 MHz, CDCl₃) δ 169.14, 139.89, 134.65, 134.19, 132.14, 129.14, 127.67, 123.95, 72.46, 46.03; MS (APCI) m/z: [M⁺-OH], 284.0; HRMS (APCI), Caclt'd for C₁₆H₁₁NClO₂ [M-OH]: 284.0473, found: 284.0481.

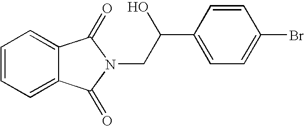

[α]=−20.3 C=1.0 in CHCl₃ (from (R)-C₃-Tunephos)

¹H NMR (300 MHz, CDCl₃) δ 7.78-7.75 (m, 2H), 7.66-7.63 (m, 2H), 7.39-7.18 (m, 4H), 5.02-4.96 (m, 1H), 4.07-3.83 (m, 2H), 2.87 (br, 1H); ¹³C NMR (75 MHz, CDCl₃) δ 168.84, 141.15, 134.22, 131.96, 128.68, 128.19, 125.96, 123.54, 72.69, 45.83; MS (APCI) m/z: [M⁺+H—OH—Br], 250.1;

HRMS (APCI), Caclt'd for C₁₆H₁₂NO₂[M+H—OH—Br]: 250.0863, found: 250.0865.

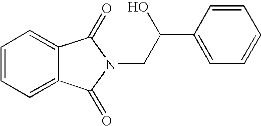

[α]=−25.6 C=1.0 in CHCl₃ (from (R)-C₃-Tunephos)

¹H NMR (300 MHz, CDCl₃) δ 7.72-7.69 (m, 2H), 7.58-7.57 (m, 2H), 7.32-7.11 (m, 5H), 4.94-4.90 (m, 1H), 3.91-3.77 (m, 2H), 2.76 (br, 1H).

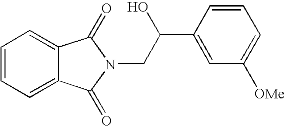

[α]=−14.9 C=1.0 in CHCl₃ (from (R)-C₃-Tunephos)

¹H NMR (300 MHz, CDCl₃) δ 7.79-7.76 (m, 2H), 7.66-7.64 (m, 2H), 7.20-7.17 (m, 1H), 6.96-6.94 (m, 2H), 6.78-6.74 (m, 1H), 4.98-4.94 (m, 1H), 3.98-3.82 (m, 2H), 3.73 (s, 3H), 2.92 (br, 1H); ¹³C NMR (75 MHz, CDCl₃) δ 169.16, 160.24, 143.12, 134.55, 132.27, 130.06, 123.87, 118.54, 114.29, 111.47, 72.97, 55.68, 46.10; MS (APCI) m/z: [M⁺−1], 280.1; HRMS (APCI), Caclt'd for C₁₇H₁₄NO₃ [M-OH]: 280.0968, found: 280.0968.

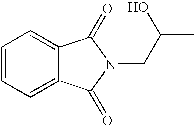

[α]=+41.3, 1.0 in CHCl₃ (from (S)-C₃-Tunephos)

¹H NMR (300 MHz, CDCl₃) δ 7.89-7.79 (m, 2H), 7.71-7.67 (m, 2H), 4.50-4.40 (m, 1H), 3.89-3.66 (m, 2H), 2.45 (br, 1H), 1.23 (d, J=6.3 Hz, 3H); ¹³C NMR (75 MHz, CDCl₃) δ 169.32, 134.51, 132.32, 123.82, 45.91, 21.48, 21.40.

The present invention has been described with particular reference to the preferred embodiments. It should be understood that the foregoing descriptions and examples are only illustrative of the invention. Various alternatives and modifications thereof can be devised by those skilled in the art without departing from the spirit and scope of the present invention. Accordingly, the present invention is intended to embrace all such alternatives, modifications, and variations that fall within the scope of the appended claims.

1. A process for preparing a non-racemic aminoalcohol, comprising:
contacting a chiral alpha-amino carbonyl compound and hydrogen, in the presence of a non-racemic hydrogenation catalyst, at a temperature, pressure and for a length of time sufficient to produce said non-racemic aminoalcohol;
wherein said chiral alpha-amino carbonyl compound is represented by formula: 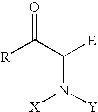
and said non-racemic aminoalcohol is represented by formula: 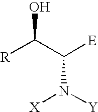
wherein R is selected from the group consisting of: hydrogen, alkyl, substituted alkyl, aryl, substituted aryl and hetereoaryl group;
E is selected from the group consisting of: hydrogen, COOR, CONHR, CONR₂, COOH, COR, CN, NO₂, alkyl, substituted alkyl, aryl, substituted aryl and hetereoaryl group; and
each X and Y is independently selected from the group consisting of: hydrogen, R, OH, NH₂, OCOR, NHCOR, POR₂, COR, COOR, CONHR and CONR₂; or wherein N, X and Y together form a cyclic imide group.

2. The process of claim 1, wherein said alpha-amino carbonyl compound is an alpha-amino ketone.

3. The process of claim 1, wherein said cyclic imide is selected from the group consisting of: phthalimide, dihydrophthalimide, tetrahydrophthalimide, succinimide, alkylsuccinimide, maleimide, alkylmaleimide and a combination thereof.

4. The process of claim 1, wherein said non-racemic hydrogenation catalyst is a non-racemic mixture of enantiomers.

5. The process of claim 1, wherein said non-racemic hydrogenation catalyst is one of the enantiomers.

6. The process of claim 1, wherein said non-racemic hydrogenation catalyst has an optical purity of at least 95% ee.

7. The process of claim 6, wherein said non-racemic hydrogenation catalyst has an optical purity of at least 85% ee.

8. The process of claim 7, wherein said non-racemic hydrogenation catalyst has an optical purity of at least 75% ee.

9. The process of claim 1, wherein said non-racemic hydrogenation catalyst is formed from a non-racemic ligand and a transition metal, a salt thereof, or complex thereof.

10. The process of claim 9, wherein said transition metal is selected from the group consisting of: Pt, Pd, Rh, Ru, Ir, Cu, Ni, Mo, Ti, V, Re and Mn.

11. The process of claim 10, wherein said transition metal is selected from the group consisting of: Pd, Rh, Ru and Ir.

12. The process of claim 10, wherein said transition metal salt, or complex thereof, is selected from the group consisting of: PtCl₂; Pd₂(DBA)₃; Pd(OAc)₂; PdCl₂(RCN)₂; (Pd(allyl)Cl)₂; (Rh(COD)Cl)₂; (Rh(COD)₂)X; Rh(acac)(CO)₂; Rh(ethylene)₂(acac); Rh(CO)₂Cl₂; Ru(RCOO)₂(diphosphine); Ru(methylallyl)₂(diphosphine); Ru(aryl group)X₂(diphosphine); RuCl₂(COD); (Rh(COD)₂)X; RuX₂(diphosphine); RuCl₂(═CHR)(PR′₃)₂; Ru(ArH)Cl₂; Ru(COD)(methylallyl)₂; Ru(arene)X₂(bisphos); Ru(RCOO)₂(bisphos); Ru(CF₃COO)₂(bisphos); Ru(methallyl)₂(bisphos); RuX₂(cymen)(bisphos); RuHX(bisphos); [Ru₂X₅(bisphos)₂]NH₂Me₂; [Ru₂X₅(bisphos)₂]NH₂Et₂; (Ir(COD)₂Cl)₂; (Ir(COD)₂)X; Cu(OTf); Cu(OTf)₂; Cu(Ar)X; CuX; NiX₂; Ni(COD)₂; MoO₂(acac)₂; Ti(OiPr)₄; VO(acac)₂; MeReO₃; MnX₂ and Mn(acac)₂; wherein each R and R′ is independently selected from the group consisting of: alkyl or aryl; Ar is an aryl group; and X is a counteranion.

13. The process of claim 12, wherein said counteranion X is selected from the group consisting of: halogen, BF₄, B(Ar)₄ wherein Ar is 3,5-di-trifluoromethyl-1-phenyl, ClO₄, SbF₆, CF₃SO₃, RCOO and a mixture thereof.

14. The process of claim 9, wherein said non-racemic hydrogenation catalyst is prepared in situ or as an isolated compound.

15. The process of claim 9, wherein said non-racemic hydrogenation catalyst is a non-racemic Ru(II) catalyst.

16. The process of claim 15, wherein said cyclic imide group is phthalimide.

17. The process of claim 16, wherein said process is represented by the reaction scheme: 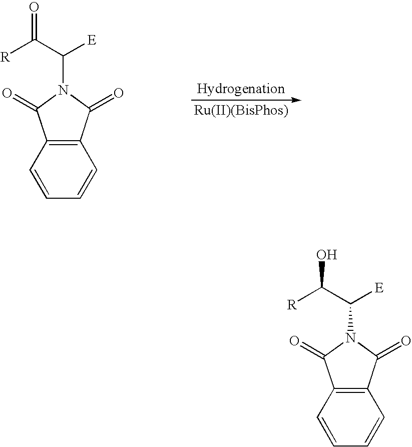
wherein R is selected from the group consisting of: hydrogen, alkyl, substituted alkyl, aryl, substituted aryl and hetereoaryl group; and
E is selected from the group consisting of: hydrogen, COOR, CONHR, CONR₂, COOH, COR, CN, NO₂, alkyl, substituted alkyl, aryl, substituted aryl and hetereoaryl group.

18. The process of claim 9, wherein said a non-racemic ligand is a non-racemic bisphosphine or diphosphine ligand selected from the group consisting of: 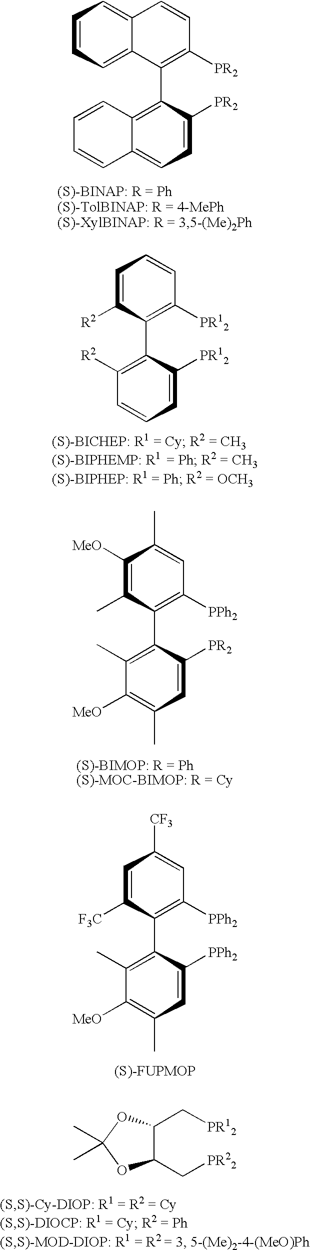 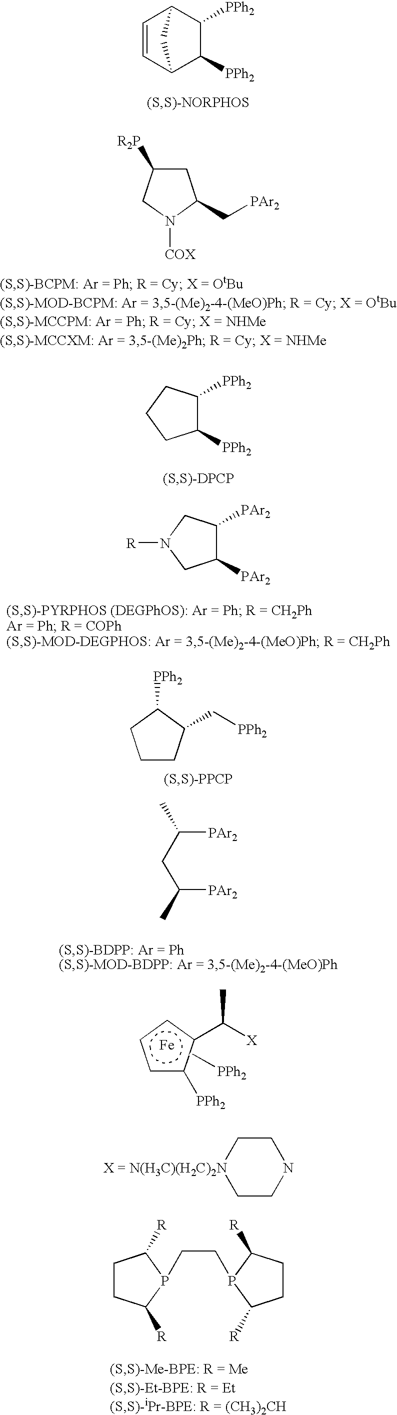 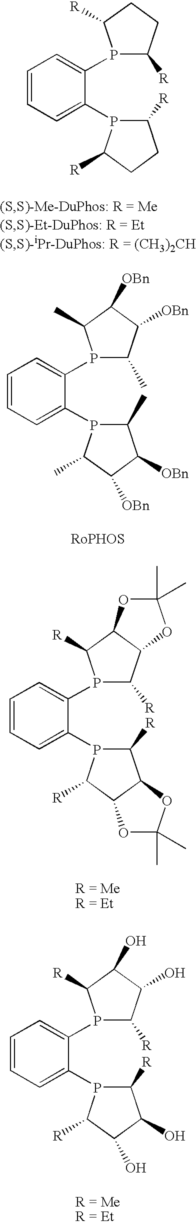 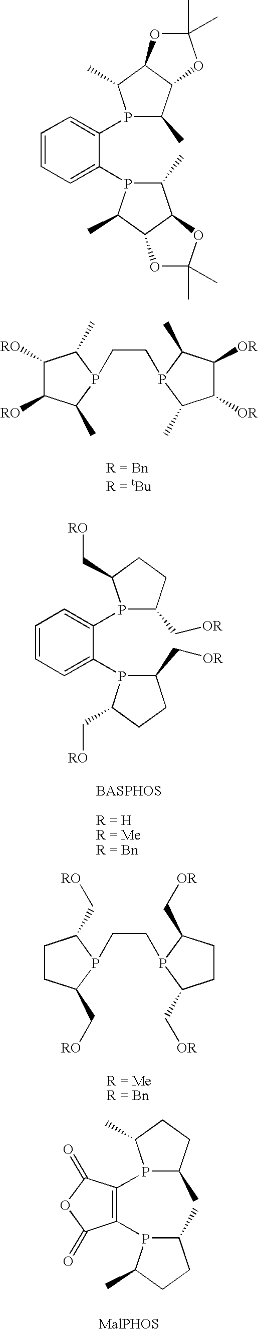 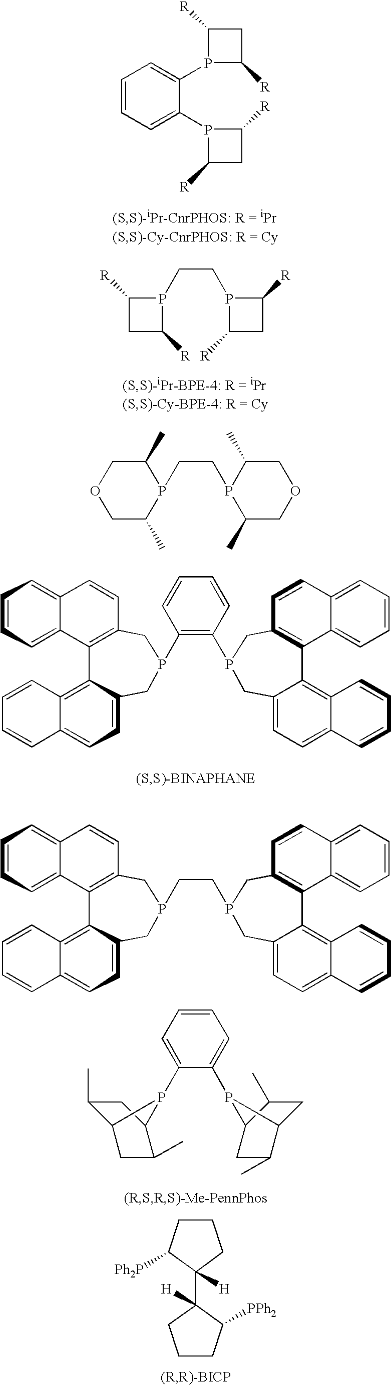 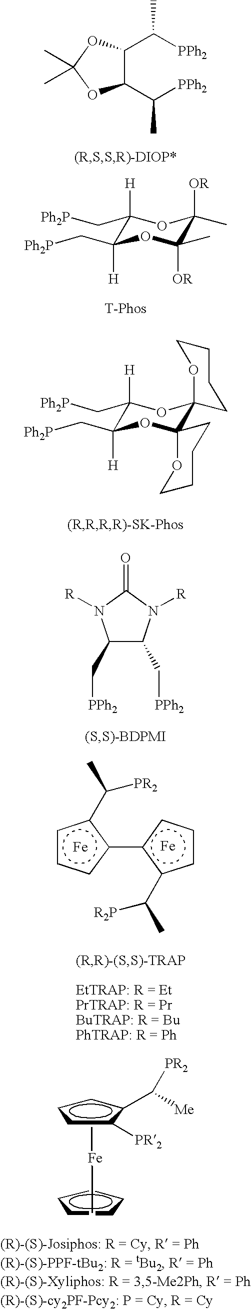 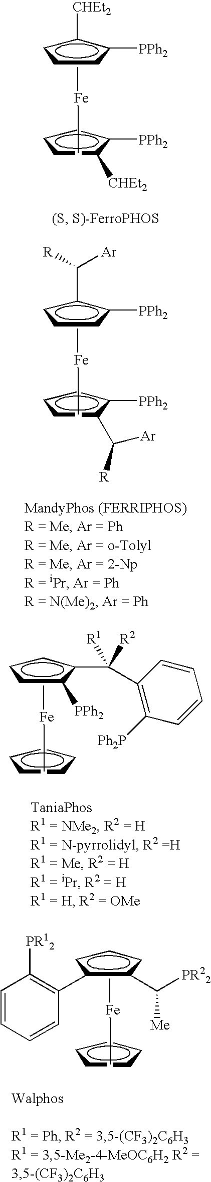 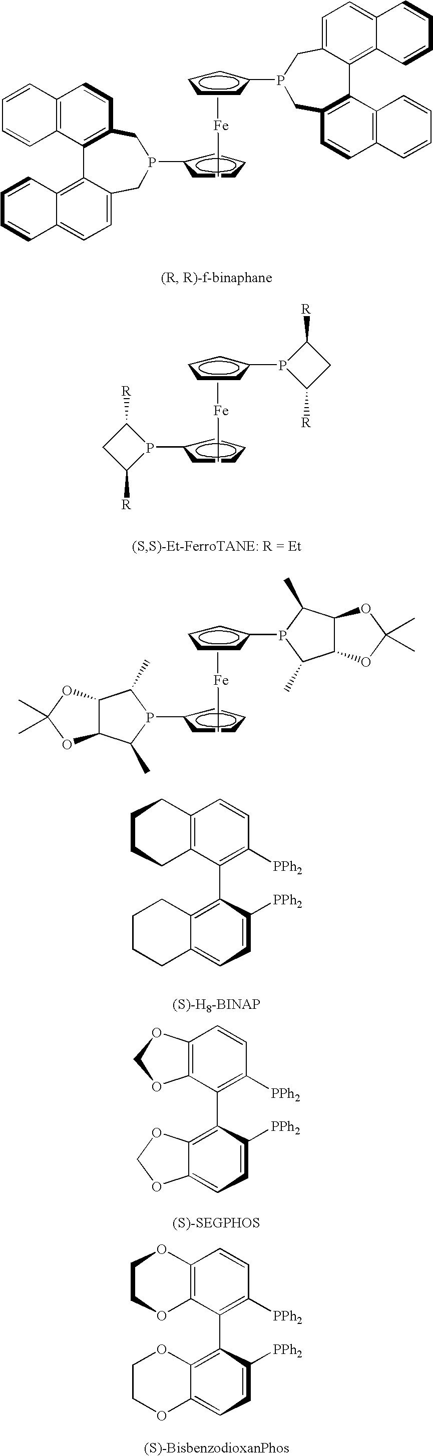 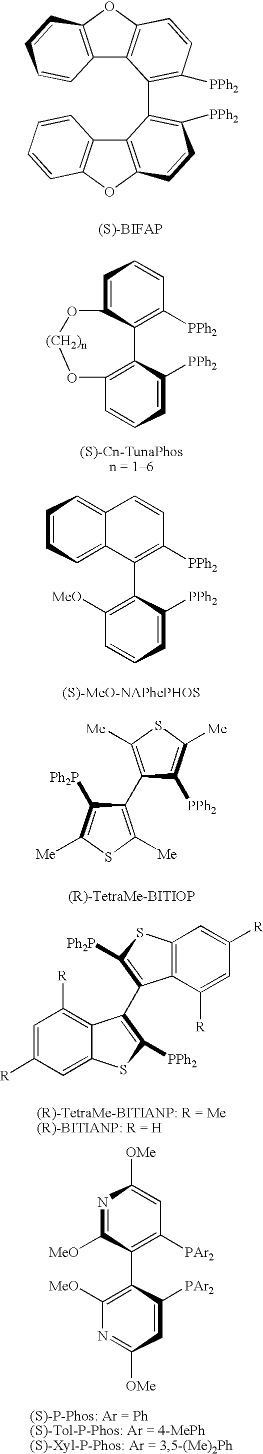 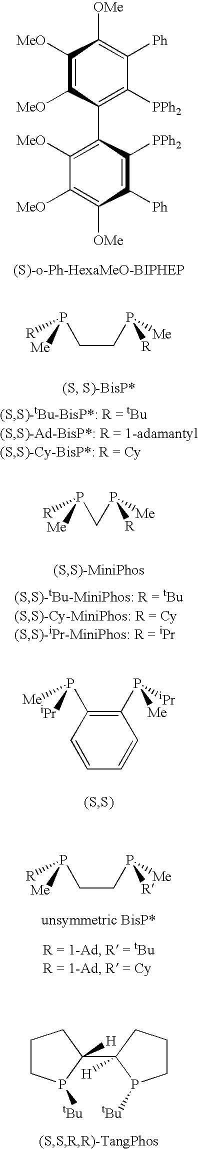 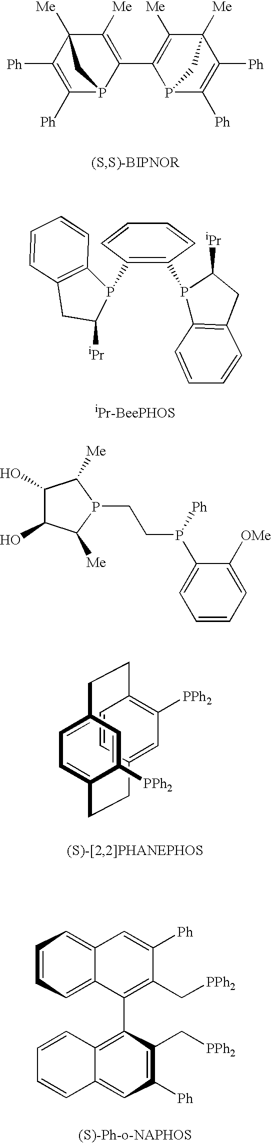
BINAP, substituted BINAP, MeO-BIPHEP, TunePhos, SEGPhos, H₈BINAP, CI-BIPHEB, MeO-BIPHEP, BIPFUP, BITIAP, BITIOP, SynPhos, P-Phos, O-BIPEP, DuPhos, Ferrotane, JesiPhos, WalPhos, MandyPhos, TaniaPhos, JafaPhos, f-KetalPhos, f-Binaphane, BPE, Rophos, ButiPhane, PennPhos, MalPhos, KetalPhos, Binaphane, BICP, DeguPhos, DIOP*, Dipamp, TangPhos, Binapine and a combination thereof.

19. The process of claim 1, wherein said non-racemic aminoalcohol formed is selected from the group consisting of compounds represented by the formula:

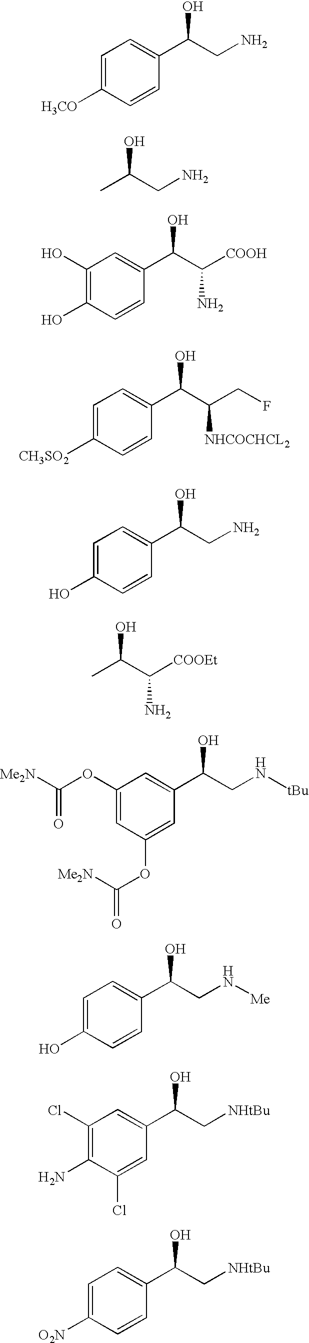

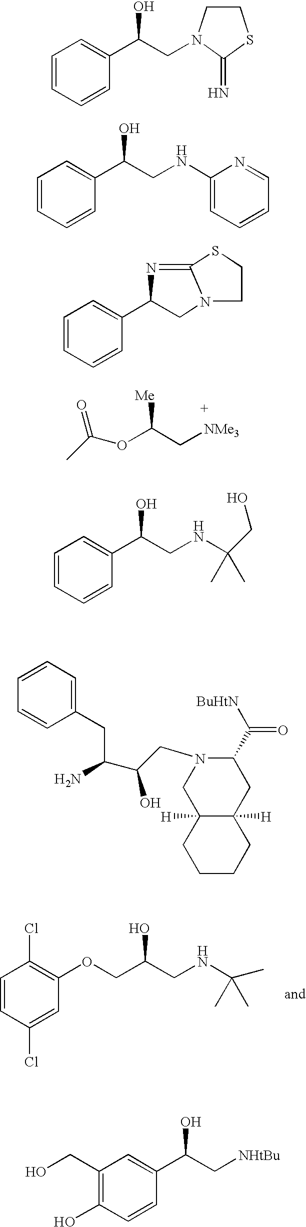

20. The process of claim 1, wherein said non-racemic aminoalcohol represented by the formula: 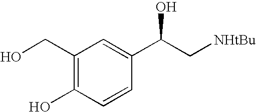
is formed from a chiral alpha-amino carbonyl compound selected from one or more compounds represented by the formula:

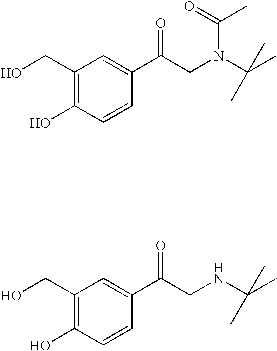

21. The process of claim 1, wherein said non-racemic hydrogenation catalyst has an optical purity of at least 98% ee.

22. The process of claim 1, wherein said non-racemic hydrogenation catalyst has an optical purity of at least 95% ee.

23. The process of claim 1, wherein said non-racemic hydrogenation catalyst has an optical purity of at least 85% ee.

24. The process of claim 7, wherein said non-racemic hydrogenation catalyst has an optical purity of at least 75% ee.
